(19) United States
(12) Patent Application Publication
(10) Pub. No.: US 2025/0136924 A1
(43) Pub. Date: May 1, 2025

(54) FOOD GRADE BACTERIA AND METHODS FOR REMOVING GLYPHOSATE AND OTHER HARMFUL SUBSTANCES

(71) Applicant: Gene Guard Detox, Inc., Ponte Vedra Beach, FL (US)

(72) Inventors: Scott Norman Peterson, Escondido, CA (US); John McColgan, North Wales, PA (US); Hans Peter Bissinger, Koili (CY); Paul J. Mills, Weaverville, NC (US)

(73) Assignee: Gene Guard Detox, Inc., Ponte Vedra Beach, FL (US)

(21) Appl. No.: 18/928,580

(22) Filed: Oct. 28, 2024

Related U.S. Application Data
(60) Provisional application No. 63/593,420, filed on Oct. 26, 2023.

(51) Int. Cl. C12N 1/20; B09B 3/60; C12R 1/07; C12R 1/225; C12R 1/25

(52) U.S. Cl. CPC C12N 1/20; B09B 3/60; C12R 2001/07; C12R 2001/225; C12R 2001/25

(57) ABSTRACT

The present disclosure relates to food-grade bacteria (GRAS isolates) and methods for removing glyphosate and other toxic compounds preventing the accumulation within organisms, feed, food and the environment. Food grade bacteria for the degradation, removal, sequestration or inactivation of glyphosate are disclosed.

This application claims priority to U.S. Provisional Patent Application No. 63/593,420, filed on Oct. 26, 2023, hereby incorporated by reference herein in its entirety.

### FIELD OF THE INVENTION

The present disclosure relates to microorganisms for removing glyphosate and other toxic compounds that have been added to the environment, feed and food supply and are potentially harmful. More particularly, the present invention relates to food grade bacteria, or extracts thereof, and to methods of using such food grade bacteria or extracts thereof to reduce the uptake of toxic and potentially harmful agricultural chemicals, and to methods of sequestering such compounds from the body or environment to which these food-grade bacteria are applied.

### BACKGROUND

Chemical pollution and its impact on health and the environment are a very complex and important issue. Thousands of man-made substances are released into the environment and can cause harm or imbalances to organisms affecting health and wellbeing. Fortunately, most of them are produced in small volumes. Some agricultural chemicals, however, are produced and applied in very large quantities leading to potential direct and indirect health hazards for animals and humans.

The full effects of synthetic chemicals on the environment, animals and humans are still poorly understood, because studies of indirect and long-term consequences are very expensive and time consuming and hence are rarely conducted (Bernhardt, E. S., Rosi, E. J., & Gessner, M. O. (2017). Synthetic chemicals as agents of global change. Frontiers in Ecology and the Environment, (2), 84-90). It remains one of the greatest problems facing humanity, and of the leading environmental causes of morbidity and mortality (Ukaogo, P. O., Ewuzie, U., & Onwuka, C. V. (2020). Environmental pollution: causes, effects, and the remedies. Microorganisms for Sustainable Environment and Health, 419-429). The main driving factors behind the increasing accumulation of potentially dangerous chemicals are hard to control macro-variables like population growth, increasing food demand and simultaneously diminishing cultivable land area. The global population has increased nearly fourfold in the past 100 years and is projected to reach 9.2 billion by 2050. Supplying food to this growing population has become a global challenge.

Large quantities of crop protection chemicals have therefore been used and are expected to be required for the foreseeable future, in order to increase food production for the growing population. The industrialization of agriculture and the extensive use of agrochemicals have indeed enhanced global food production, a transformation that has fundamentally altered earth and has led to substantial environmental degradation of soil and water quality worldwide (Vitousek, P. M., Mooney, H. A., Lubchenco, J., & Melillo, J. M. (1997). Human domination of Earth's ecosystems. Science, 277 (5325), 494-499; Cordell, D., Drangert, J. O., & White, S. (2009). The story of phosphorus: global food security and food for thought. Global environmental change, 19 (2), 292-305.).

Although fertilizer use dropped notably in many areas of North America in the 1980s and 1990s (e.g. Eastern Canada, Northeastern US) (MacDonald, G. K., & Bennett, E. M. (2009). Phosphorus accumulation in Saint Lawrence River watershed soils: a century-long perspective, Ecosystems, 12 (4), 621-635; Hale, R. L., Hoover, J. H., Wollheim, W. M., & Vörösmarty, C. J. (2013). History of nutrient inputs to the northeastern United States, 1930-2000. Global Biogeochemical Cycles, 27 (2), 578-591), at the global scale herbicide and pesticide use has risen dramatically in that timeframe (Gilbert, N. (2013). Case studies: A hard look at GM crops, Nature News, 497 (7447), 24). In 2019, the agrochemical market worldwide was valued at 243.1 billion U.S. dollars. This is expected to increase to nearly 300 billion U.S. dollars by 2024.

Herbicides, one of the major groups of agrochemicals are used to kill or inhibit the growth of unwanted plants, such as residential or agricultural weeds and invasive species. The ease of application is a big advantage of chemical herbicides over mechanical weed control, which often saves on labor costs and preserves soil via no tilling practice. The global herbicides market is estimated at USD 27.21 billion in 2016 and is projected to reach USD 39.15 billion by 2022, at a CAGR of 6.25% during the forecast period. Herbicides are one of the major crop protection chemicals used for weed control.

The herbicides market is segmented based on herbicide type, mode of action, crop type, and region. On the basis of type, the herbicide market was dominated by the glyphosate segment and was followed by diquat and 2,4-D segments in 2015. With increasing use of glyphosate-based products in various forms, such as gel and powder across countries, glyphosate is projected to remain the leading herbicide.

As a non-selective herbicide, glyphosate is formulated for both broadleaf and grass weeds, and it finds application in most of the vegetation types and is a preferred alternative to selective herbicides. Additionally, with the introduction of genetically modified crops that have herbicide tolerance, the demand for non-selective herbicides is projected to further increase. Glyphosate has become the most widely used agricultural chemical in history due to its ability to enhance food production and a relative lack of previous concern about its persistence and toxicity as compared to other types of herbicides.

### Glyphosate

Glyphosate—a phosphonic acid with the formula C₃H₈NO₅P, also known as N-(phosphonomethyl) glycine—was introduced as a broad-spectrum herbicide in 1974, and it quickly became one of the most heavily used herbicides worldwide (Nadin, P. (2007). The use of plant protection products in the European union data 1992-2003. European Commission: Luxembourg, France). With the introduction of genetically engineered glyphosate-tolerant crops, glyphosate use increased dramatically in the late-1990s and 2000s. In addition to agricultural uses, glyphosate is one of the most common residential herbicides in the United States. By 2010, more than 750 products containing glyphosate were on the U.S. market, and it was registered for use in more than 130 countries. Glyphosate went off-patent in the 1990s, prompting the development of generic herbicide formulations that are less costly and consequently more accessible, including in developing countries.

Approximately 8.6 billion kg of glyphosate have been applied globally since 1974 (Benbrook, C. M. (2016). Trends in glyphosate herbicide use in the United States and globally. Environmental Sciences Europe, 28 (1), 1-15); 1.6 billion kg of which has been applied in the U.S. alone, with two-thirds of this amount having been applied in just the past 10 years. Glyphosate-based herbicides are predominantly used in association with genetically engineered glyphosate resistant crops (GRCs), especially the genetically modified corn (Zea mays), soybean (Glycine max), and cotton (Gossypium spp.) varieties that form the so-called “ROUNDUP® Ready” crops first introduced in 1996 in the U.S. (Duke, S. O., & Powles, S. B. (2008). Glyphosate: a once-in-a-century herbicide. Pest Management Science: formerly Pesticide Science, 64 (4), 319-325). The widespread adoption of GRCs (e.g. in the U.S., Brazil, and Argentina) has increased glyphosate application by ˜15-fold, with GRC cultivation currently accounting for 56% of global glyphosate use (Givens, W. A., Shaw, D. R., Kruger, G. R., Johnson, W. G., Weller, S. C., Young, B. G., . . . & Jordan, D. (2009). Survey of tillage trends following the adoption of glyphosate-resistant crops. Weed technology, 23 (1), 150-155; Benbrook, C. M. (2016). Trends in glyphosate herbicide use in the United States and globally. Environmental Sciences Europe, 28 (1), 1-15). The combination of the broad-spectrum nature of glyphosate, the introduction of GRCs, and increasingly intensive farming practices (e.g. no-till cropping, monocultures) has elevated the class of glyphosate-based herbicides to the degree that they have become the most applied agricultural chemical in human history.

Glyphosate usage is heaviest in the Midwest due to corn and soybean production. Agricultural run-off has resulted in high detection rates of glyphosate in US streams, rivers, and lakes (Struger, J., Thompson, D., Staznik, B., Martin, P., McDaniel, T., & Marvin, C. (2008). Occurrence of glyphosate in surface waters of southern Ontario. Bulletin of Environmental Contamination and Toxicology, 80 (4), 378-384). In addition, crops that are genetically modified to be glyphosate resistant (i.e., ROUNDUP®-ready) have glyphosate residue. Over 90% of corn, soy, and canola grown in the United States are modified in this way, and these grains are used in most processed foods (Dodson, L. (2020). Use of Genetically Engineered Cotton Has Shifted Toward Stacked Seed Traits. Amber Waves: The Economics of Food, Farming, Natural Resources, and Rural America, 2020 (1490-2020-1812); USDA.gov/data-products/adoption-of-genetically-engineered-crops-in-the-us/recent-trends-in-ge-adoption.aspx. accessed 10 May 2021). Glyphosate use has risen almost 15-fold since so-called “ROUNDUP® Ready,” genetically engineered glyphosate-tolerant crops were introduced in 1996 (Benbrook, C. M. (2016). Trends in glyphosate herbicide use in the United States and globally. Environmental Sciences Europe, 28 (1), 1-15), and sales are projected to rise by a further 25% by 2024. U.S. American farmers sprayed 290 million pounds of glyphosate on their crops in 2016, according to U.S. Geological Survey data. That amounts to nearly a pound of glyphosate for every person in the country.

### The Changing View on Glyphosate Safety

The previously prevalent but gradually changing view that glyphosate has a low risk of adverse health or toxicological impacts on animals and humans has led to decades of permissive regulation of its use, and in some cases maximum exposure thresholds tolerated in aquatic ecosystems have actually been revised upward (e.g. in Canada).

One reason for this was its relatively low immediate toxicity in the established assays due to the absence of its targeted biochemical pathway in animals and humans. However there still exists a lack of comprehensive studies and understanding about its effects on the environment or people's health. All the scientific production linked to glyphosate analyzed from the period from 1974 to 2016, using the Web of Science (WOS) as a source of information and “glyphosate” as a search criterion shows a record of 8,174 publications. There was a concentration in knowledge generation led by the United States. Monsanto Company produced the largest number of articles during the first thirty years of the development of this topic. Research around glyphosate in South America gained importance as of the year 2000, with Brazil being the country with the highest production followed by Argentina. A large proportion of research was focused on agricultural science, while an in-depth analysis of toxicology or environmental effects did not begin until the year 2000, and is still incomplete in many aspects. (Sosa, B., Fontans-Álvarez, E., Romero, D., da Fonseca, A., & Achkar, M. (2019). Analysis of scientific production on glyphosate: An example of politicization of science. Science of the Total Environment, 681, 541-550).

Glyphosate interferes with the synthesis of aromatic amino acids by inhibiting the enzyme 5-enolpyruvylshikimate-3-phosphate synthase, which is responsible for biosynthesis of the aromatic amino acids: phenylalanine, tyrosine, and tryptophan via the shikimate pathway, a mechanism specific to plants and certain bacteria. In its 1993 re-registration decision, the U.S. Environmental Protection Agency determined that there were no “unreasonable risks or adverse effects to humans or the environment” and indicated that all uses were eligible for registration.

### Glyphosate Affects Non-Target Organisms.

The view on glyphosate took a sharp turn when, in March 2015, the International Agency for Research on Cancer (IARC), which is the specialized cancer agency of the World Health Organization (WHO), classified glyphosate as ‘probably carcinogenic to humans’, while regulatory authorities worldwide have attributed non-carcinogenic properties to the substance and have authorized its use as an active substance in pesticides.

Long term exposure has been linked to diseases such as Parkinson's, asthma, and leukemia. (Suppa, A., Kvist, J., Li, X., Dhandapani, V., Almulla, H., Tian, A. Y., . . . & Orsini, L. (2020). ROUNDUP® causes embryonic development failure and alters metabolic pathways and gut microbiota functionality in non-target species. Microbiome, 8 (1), 1-15) Glyphosate containing ROUNDUP® causes embryonic developmental failure and alters metabolic pathways and gut microbiota functionality in non-target species (Mao, Q., Manservisi, F., Panzacchi, S., Mandrioli, D., Menghetti, I., Vornoli, A., . . . & Hu, J. (2018). The Ramazzini Institute 13-week pilot study on glyphosate and ROUNDUP® administered at human-equivalent dose to Sprague Dawley rats: effects on the microbiome. Environmental Health, 17 (1), 1-12).

In utero exposure to either ROUNDUP® or glyphosate alone has been linked to birth defects and fetal loss in chickens, frogs, and mammals (Parvez, S., Gerona, R. R., Proctor, C., Friesen, M., Ashby, J. L., Reiter, J. L., . . . & Winchester, P. D. (2018). Glyphosate exposure in pregnancy and shortened gestational length: a prospective Indiana birth cohort study. Environmental Health, 17 (1), 1-12).

While these studies did not replicate human exposure in the low dose range, they found fetal toxicity risks such as dilated hearts and visceral anomalies in rats and post-implantation loss and late embryonic deaths in rabbits at doses as low as 20 mg/kg/day. Glyphosate also has been shown to disrupt important pathways in development, such as retinoic acid signaling and estrogen biosynthesis (Paganelli, A., Gnazzo, V., Acosta, H., López, S. L., & Carrasco, A. E. (2010). Glyphosate-based herbicides produce teratogenic effects on vertebrates by impairing retinoic acid signaling. Chemical research in toxicology, 23 (10), 1586-1595). Further, glyphosate has been reported to disrupt enzymatic pathways, such as the cytochrome P450, and to damage DNA structure in human breast epithelial and placental cells (Richard, S., Moslemi, S., Sipahutar, H., Benachour, N., & Seralini, G. E. (2005). Differential effects of glyphosate and ROUNDUP® on human placental cells and aromatase. Environmental health perspectives, 113 (6), 716-720; Gasnier, C., Dumont, C., Benachour, N., Clair, E., Chagnon, M. C., & Séralini, G. E. (2009). Glyphosate-based herbicides are toxic and endocrine disruptors in human cell lines. Toxicology, 262 (3), 184-191; Benachour, N., Sipahutar, H., Moslemi, S., Gasnier, C., Travert, C., & Seralini, G. E. (2007). Time- and dose-dependent effects of ROUNDUP® on human embryonic and placental cells. Archives of environmental contamination and toxicology, 53 (1), 126-133).

Glyphosate inhibits aromatase (CYP19A1) activity by a direct interaction with the active site of the enzyme at concentrations 100 times lower (0.036 g/L) than the recommended use in agriculture, i.e., 3.6 g/L (Richard, S., Moslemi, S., Sipahutar, H., Benachour, N., & Seralini, G. E. (2005). Differential effects of glyphosate and ROUNDUP® on human placental cells and aromatase. Environmental health perspectives, 113 (6), 716-720). Moreover, glyphosate induced DNA damage and chromosomal breaks in vitro and in vivo in mice (Bolognesi, C., Bonatti, S., Degan, P., Gallerani, E., Peluso, M., Rabboni, R., and Abbondandolo, A. (1997). Genotoxic activity of glyphosate and its technical formulation ROUNDUP®. Journal of Agricultural and food chemistry, 45 (5), 1957-196).

Despite evidence of potential genotoxicity and teratogenic activity of glyphosate in animal studies, glyphosate effects on human pregnancy and fetal development have not been investigated. Epidemiological evidence of glyphosate exposure effects on reproductive and developmental health outcomes is limited. A systematic review conducted in 2016 found only ten studies that had tested the association between indirect measure of glyphosate exposure and adverse pregnancy outcomes (de Araujo, J. S., Delgado, I. F., & Paumgartten, F. J. (2016). Glyphosate and adverse pregnancy outcomes, a systematic review of observational studies. BMC Public Health, 16 (1), 1-13). A California-based study on a rural pregnant population for their residential proximity to farmland and exposure during the pre- and post-conception period (i.e., 4 weeks before and 8 weeks after conception) reported an increased risk of neural tube defects (Odds ratio (OR)=1.5; 95% confidence interval (CI), 1.0-2.4. (Rull, R. P., Ritz, B., & Shaw, G. M. (2006). Validation of self-reported proximity to agricultural crops in a case-control study of neural tube defects. Journal of exposure science & environmental epidemiology, 16 (2), 147-155). A Minnesota Red River Valley study reported a significant association between male perinatal exposure to multiple pesticides including glyphosate with increased risk (OR=3.6; 95% CI, 1.0-4.0) of attention deficit hyperactivity disorder (Garry, V. F., Schreinemachers, D., Harkins, M. E., & Griffith, J. (1996). Pesticide appliers, biocides, and birth defects in rural Minnesota. Environmental health perspectives, 104 (4), 394-39; Garry, V. F., Harkins, M. E., Erickson, L. L., Long-Simpson, L. K., Holland, S. E., & Burroughs, B. L. (2002). Birth defects, season of conception, and sex of children born to pesticide applicators living in the Red River Valley of Minnesota, USA. Environmental health perspectives, 110 (suppl 3), 441-449). In addition, a few reproductive epidemiological studies have evaluated glyphosate's association with non-congenital anomalies with mostly negative results. These include the Canadian Ontario Farm Family Health Study (Weselak, M., Arbuckle, T. E., Wigle, D. T., Walker, M. C., & Krewski, D. (2008). Pre- and post-conception pesticide exposure and the risk of birth defects in an Ontario farm population. Reproductive Toxicology, 25 (4), 472-480; Weselak, M., Arbuckle, T. E., & Foster, W. (2007). Pesticide exposures and developmental outcomes: the epidemiological evidence. Journal of Toxicology and Environmental Health, Part B, 10 (1-2), 41-80; Arbuckle, T. E., Lin, Z., & Mery, L. S. (2001). An exploratory analysis of the effect of pesticide exposure on the risk of spontaneous abortion in an Ontario farm population. Environmental health perspectives, 109 (8), 851-857; Wigle, D. T., Arbuckle, T. E., Turner, M. C., Bérubé, A., Yang, Q., Liu, S., & Krewski, D. (2008). Epidemiologic evidence of relationships between reproductive and child health outcomes and environmental chemical contaminants. Journal of Toxicology and Environmental Health, Part B, 11 (5-6), 373-517), a Columbian Study, and the Iowa and North Carolina Farm and Nonfarm Family Studies (Sanin, L. H., Carrasquilla, G., Solomon, K. R., Cole, D. C., & Marshall, E. J. P. (2009). Regional differences in time to pregnancy among fertile women from five Colombian regions with different use of glyphosate. Journal of Toxicology and Environmental Health, Part A, 72 (15-16), 949-960; Sathyanarayana, S., Basso, O., Karr, C. J., Lozano, P., Alavanja, M., Sandler, D. P., & Hoppin, J. A. (2010). Maternal pesticide use and birth weight in the agricultural health study. Journal of agromedicine, 15 (2), 127-136).

Only the Ontario Farm Family Health study reported significant association between perinatal exposure to glyphosate (and other pesticides) and increased risk (OR=1.7; 95% CI, 1.0-1.6) of spontaneous abortion later in pregnancy (12-19 weeks). Thus, most prior epidemiological studies have been of limited sizes and findings have been inconclusive due to methodological limitations, lack of direct measurement of glyphosate, and without definitive evidence that glyphosate exposure harms human fetal development. Despite these inconclusive epidemiological studies, the prevalence of glyphosate residues in genetically modified crops and contaminated drinking water (Duke & Powles, 2008 supra; WHO, World Health Organization: Pesticide residues in food. FAO Plant Production and Protection Paper 2005; (182/1): 1-273; Woodburn, A. T. (2000). Glyphosate: production, pricing and use worldwide. Pest Management Science: formerly Pesticide Science, 56 (4), 309-312; Benbrook 2016 supra; Coupe, R. H., Kalkhoff, S. J., Capel, P. D., & Gregoire, C. (2012). Fate and transport of glyphosate and aminomethylphosphonic acid in surface waters of agricultural basins. Pest management science, 68 (1), 16-30) warrants further investigation to determine the risk of adverse fetal outcomes due to glyphosate exposure.

More recent animal and human studies, however, suggest that chronic exposure to glyphosate-based herbicides can induce adverse health outcomes. (Myers, J. P., Antoniou, M. N., Blumberg, B., Carroll, L., Colborn, T., Everett, L. G., . . . & Benbrook, C. M. (2016). Concerns over use of glyphosate-based herbicides and risks associated with exposures: a consensus statement. Environmental Health, 15 (1), 1-13). Animals consistently fed an ultra-low dosage of the herbicide with a 50-ng/L glyphosate concentration show hepatotoxicity consistent with nonalcoholic fatty liver disease and its progression to steatohepatosis (Mesnage, R., Renney, G., Séralini, G. E., Ward, M., & Antoniou, M. N. (2017). Multiomics reveal non-alcoholic fatty liver disease in rats following chronic exposure to an ultra-low dose of ROUNDUP® herbicide. Scientific reports, 7 (1), 1-15). In July 2017, in accordance with the Safe Drinking Water and Toxic Enforcement Act of 1986, the state of California listed glyphosate as a probable carcinogen.

Major routes of public exposure are through consumption via diet. Agricultural workers and industrial workers are at increased risk of exposure through workplace by absorption or inhalation if safety protocols are not properly followed.

The increasing use of glyphosate in huge quantities has resulted in several other unforeseen issues, such as the emergence of glyphosate-resistant weeds, and changes to the human gut microbiome via the inhibition of 5-enolpyruvylshikimate-3-phosphate synthase of the shikimate pathway in the gut microbiome (Mesnage, et al. (2017) supra). Multiomics reveal non-alcoholic fatty liver disease in rats following chronic exposure to an ultra-low dose of ROUNDUP® herbicide. Scientific reports, 7 (1), 1-15).

Animals and humans do indeed lack the shikimate pathway, which is why glyphosate was initially considered one of the least toxic pesticides used in agriculture (Van Straalen, N. M., & Legler, J. (2018). Decision-making in a storm of discontent. Science, 360 (6392), 958-960). However, some evidence suggests that glyphosate affects nontarget organisms, for example, reducing reproduction of soil-dwelling earthworms (Clausing, P. (2019). Glyphosate: The European Controversy-A Review of Civil Society Struggles and Regulatory Failures. Business and Human Rights Journal, 4 (2), 351-356) and affecting the growth of microalgae and aquatic bacteria (Landrigan, P. J., & Belpoggi, F. (2018). The need for independent research on the health effects of glyphosate-based herbicides. Environmental Health, 17 (1), 1-4). Glyphosate is also associated with changes in plant endophytic and rhizosphere microbiomes (Cuhra, M. (2018). Evolution of glyphosate resistance: Is the rhizosphere microbiome a key factor. J. Biol. Phys. Chem, 18, 78-93) and with disturbances of gut microbiota of animals living near agricultural sites (Richmond, M. E. (2018). Glyphosate: a review of its global use, environmental impact, and potential health effects on humans and other species. Journal of Environmental Studies and Sciences, 8 (4), 416-434). Researchers recently found a range of negative impacts on honeybees exposed to glyphosate-based pesticides at or below recommended concentrations (Motta, E. V., Raymann, K., & Moran, N. A. (2018). Glyphosate perturbs the gut microbiota of honey bees. Proceedings of the National Academy of Sciences, 115 (41), 10305-10310).

As glyphosate targets the 5-enolpyruvylshikimate-3-phosphate synthase (EPSPS) enzyme in the shikimate pathway found in plants and some microorganisms, glyphosate may affect bacterial symbionts of animals living near agricultural sites, including pollinators such as bees. The honey bee gut microbiota is dominated by eight bacterial species that promote weight gain and reduce pathogen susceptibility. It was demonstrated that the relative and absolute abundances of dominant gut microbiota species are decreased in bees exposed to glyphosate at concentrations documented in the environment. Glyphosate exposure of young worker bees increased mortality of bees exposed to the opportunistic pathogen Serratia marcescens. Bees deprived of their normal microbiota show reduced weight gain and altered metabolism (Zheng, H., Powell, J. E., Steele, M. I., Dietrich, C., & Moran, N. A. (2017). Honeybee gut microbiota promotes host weight gain via bacterial metabolism and hormonal signaling. Proceedings of the National Academy of Sciences, 114 (18), 4775-4780), increased pathogen susceptibility, and increased mortality within hives (Motta, E. V., & Moran, N. A. (2020). Impact of glyphosate on the honeybee gut microbiota: effects of intensity, duration, and timing of exposure. Msystems, 5 (4)).

Thus, exposure of bees to glyphosate clearly can perturb their beneficial gut microbiota, potentially affecting bee health and their effectiveness as pollinators.

Many scientific debates have therefore arisen regarding the direct and indirect effects of glyphosate use on the environment and human health (Benbrook, C. M. (2019). How did the U.S. EPA and IARC reach diametrically opposed conclusions on the genotoxicity of glyphosate-based herbicides?. Environmental Sciences Europe, 31 (1), 1-16) and regarding glyphosate registration and regulations (Van Straalen & Legler, 2018 supra; Székács, A., & Darvas, B. (2018). Re-registration challenges of glyphosate in the European Union. Frontiers in Environmental Science, 6, 78; Clausing, 2019 supra; Landrigan & Belpoggi 2018 supra; Richmond, 2018 supra), and have led, for example, to a European Citizens' Initiative to ban glyphosate and protect people and the environment from toxic pesticides (Citizens Initiative ECI (2017) 000002. 2017. Available online (accessed on 2 May 2021)), as well as the decision by the Austrian parliament to ban glyphosate (Murphy, F.; Schwarz-Goerlich, A., Austrian Parliament Backs EU's First Total Ban of Weedkiller glyphosate; Reuters: Vienna, Austria, 2019. Available online: www.reuters.com/article/us-austria-glyphosate/austrian-parliamentbacks-eus-first-total-ban-of-weedkiller-glyphosate-idUSKCN1TX1JR (accessed on 23 Feb. 2021); Peng, W., Lam, S. S., & Sonne, C. (2020). Support Austria's glyphosate ban. Science, 367 (6475), 257-258), and a statement by the German government that glyphosate use should be significantly reduced by 2023 (Kudsk, P., & Mathiassen, S. K. (2020). Pesticide regulation in the European Union and the glyphosate controversy. Weed Science, 68 (3), 214-222).

### Phthalates are Ubiquitous in the Environment and Human Systems Negatively Impacting Human Health.

Phthalates represent general-purpose plasticizers used in the plastic industry, especially polyvinyl chloride (PVC), while bisphenol A (BPA) plays an important role as one of the raw materials in the manufacture of polycarbonates, polystyrene, and epoxy resins. Phthalates are the most commonly used plasticizers in the market at present and can reach 20-50% of the final product. Importantly, phthalates and bisphenol A are not covalently bound to the plastic, and they are constantly released into the environment by migration, evaporation, leaching and abrasion from consumer products. With time, such substances accumulate through the food chain and are difficult to degrade in the natural environment. Di-butyl phthalate (DBP), one of the plasticizers, is most commonly used in polyvinyl chloride (PVC) processing, which can lead to reproductive tract malformation in male rats during sexual differentiation (Howdeshell et al. (2007), Cumulative effects of dibutyl phthalate and diethylhexyl phthalate on male rat reproductive tract development: altered fetal steroid hormones and genes. Toxic. Sci. 99 (1) 190-202), affecting the secretion of human sex hormones and threatening human health (Ghisari and Bonefeld-Jorgensen (2009), Effects of plasticizers and their mixtures on estrogen receptor and thyroid hormone functions. Toxicol Lett 18 (1) 67-77). Meanwhile, DBP was proved to be harmful to rodents (Higuchi et al., (2003), Effects of dibutyl phthalate in male rabbits following in utero, adolescent, or postpubertal exposure. Toxicol Sci 72 (2) 301-313), and further studies showed that DBP could induce antiandrogenic effects by inhibiting steroidogenic factor 1 (SF1) indirectly (Plummer et al., (2013), Identification of transcription factors and coactivators affected by dibutylphthalate interactions in fetal rat testes. Toxicol Sci 132 (2) 443-457). Some research demonstrated that the exposure to DBP disrupted ovarian function in animal models and in human cells in vitro (Adir et al. (2017), Dibutyl phthalate impairs steroidogenesis and a subset of LH-dependent genes in cultured human mural granulosa cell in vitro. Reprod Toxicol. 69 (1) 13-18).

Phthalates and bisphenol A (BPA) have drawn the attention of the scientific community, regulatory agencies and general public due to their widespread use, omnipresence in the environment, and growing concern for their impact on human health. Because of their comprehensive use as important components of multiple consumer products, as well as in industrial processes, and consequential ubiquitous presence in the environment, human exposure to these substances is almost inevitable. Phthalate metabolites have mostly been measured in urine, but also serum, amniotic fluid, and saliva. Human biomonitoring studies have shown that metabolites of diethyl phthalate (DEP), dibutyl phthalate (DBP), diisobutyl phthalate (DiBP), and bis(2-ethylhexyl) phthalate (DEHP) were the most abundant compounds detected in urine (Wang et al., (2019), A review of Biomonitoring of Phthalate Exposures. Toxics 7 (2) 21). On the other hand, bisphenol A has been measured in different biological matrices, such as urine, human cord blood, placenta, amniotic fluid and breast milk. Phthalate acid esters (PAEs) and Ortho-PAEs have substantial migration potential and are present at high concentrations in these products (Hildenbrand et al. (2019), Inter-day variability of metabolites of DEHP and DnBP in human urine-comparability of the results of longitudinal studies with a cross-sectional study. Int J Environ Res Public Health 16 (6) 1029). Intake of PAEs occurs through water, food, air, soil and dust via ingestion, inhalation, or absorption, and they can work as endocrine-disrupting chemicals (EDCs) and interfere with hormones (Braun, et al., (2016), Effects of Environmental Exposures on Fetal and Childhood Growth Trajectories Ann. Glob. Health 82 (1) 41-99). Di-n-butyl phthalate (DBP) is widely used in epoxy resins, cellulose esters, and in special adhesive formulations (He et al. (2015), Monitoring of Phthalates in Foodstuffs Using Gas Purge Microsyringe Extraction Coupled with GC-MS Anal Chim Acta (879) 63-68). DBP is known to be teratogenic in animals (Giribabu & Reddy (2017), Protection of male reproductive toxicity in rats exposed to di-n-butyl phthalate during embryonic development by testosterone Biomed Pharmacother 87, 355-365). Hsieh et al., (2019), Personal care products use and phthalate exposure levels among pregnant women Sci Total Environ 648, 135-143) found in a multi-hospital-based birth cohort that fetuses were susceptible to phthalates derived from the personal care products (PCPs) used by women during pregnancy. The daily tolerable intake for DBP as specified by the European Food Safety Authority is 0.01 mg/kg body weight. Thus, it is urgent to develop an efficient method for DBP removal.

Over the past few decades, lactic acid bacteria (LAB), e.g. Lactobacillus acidophilus, strains have been attributed health-promoting effects on the host, and can lower genotoxic and carcinogenic toxicity, and reduce the risk of carcinogenic compounds by removing them using various toxin-binding structures (Sanders et al., (2014), Mitochondrial DNA damage as a peripheral biomarker for mitochondrial toxin exposure in rats Toxicol Sci 142 (2) 395-402); Wacoo et al., (2019), Probiotic enrichment and reduction of Aflotoxins in a traditional African maize-based fermented food. Nutrients 11 (2) 265). A noncovalent interaction has been reported between the carcinogenic compounds and the carbohydrate or protein moieties in the bacterial cell wall during the binding process, and the structural integrity of the bacterial cell wall is required for adsorption (Zhang et al., (2016), Human CYP2E1-dependent mutagenicity of mono- and dichlorobiphenyls in Chinese hamster (V79)-derived cells. Chemosphere 144, 1908-1915). Peptidoglycan and teichoic acid are generally recognized as the major components in Gram-positive bacterial cell walls (Nygaard et al., (2015), Spectral snapshots of bacterial cell-wall composition and the influence of antibiotics by whole-cell NMR. Biophys. J. 108 (6) 1380-1389). The primary component and structural scaffold of the cell wall is peptidoglycan, comprising a repeating unit of N-acetylglucosamine (NAG) and N-acetylmuramic disaccharide (NAM) [NAG-(β-1,4)-NAM] with a peptide attached to the D-lactyl moiety of each NAM (Meroueh et al., (2006), Three-dimensional structure of the bacterial cell wall peptidoglycan Proc. Natl. Acad. Sci USA 103 (12) 4404-4409). For instance, the peptidoglycan molecular scaffold of Lactobacillus acidophilus consists of a main chain of NAG-(β-1,4)-NAM-L-Ala-D-Glu-L-Lys-D-Ala (Wu et al., (2013), Structure and anti-inflammatory capacity of peptidoglycan from Lactobacillus acidophilus in RAW-264.7 cells. Carbohyd. Polym 96 (2) 466-473), subunits comprising the disaccharide, pentapeptide stem, and the L-Lys-D-Asp sequence of peptide chain presumably linked to its main chain (Schleifer et al., (1972), Peptidoglycan types of bacterial cell walls and their taxonomic implications. Bacteriol Rev 36 (4) 407-477; Coyette et al. (1970), Structure of the walls of Lactobacillus acidophilus strain 63 AM Gasser. Biochemistry 9 (15) 2935-2943), indicated that the bisdisaccharide peptide dimer represented the maximum fraction of bacterial peptidoglycan. Teichoic acids are attached to peptidoglycan, and 32.80% of the cell wall content in L. acidophilus is composed of teichoic acid (Sagmeister, et al., (2024), The molecular architecture of Lactobacillus S-layer: Assembly and attachment to teichoic acids. Proc. Natl. Acad. Sci USA 121 (24)). Thus, it is feasible to visualize a peptidoglycan model of L. acidophilus with a bisdisaccharide peptide dimer and teichoic acid structure combined with the peptidoglycan dimer in the cell wall model. However, due to the complexity of cell wall composition, the critical cell wall structures and binding sites involved in removing carcinogenic compounds are still unclear.

Exposure to PAEs occurs through the intaking of chemicals via ingestion, inhalation or skin absorption (Karamianpour J., et al., (2023), Accumulation, sources and health risks of phthalic acid esters (PAEs) in road dust from heavily industrialized, urban and rural areas in Southern Iran Heliyon 9 (12)). PAEs can interfere with human body's normal hormones secretion and produce various toxic effects. Especially, Di-n-butyl phthalate (DBP), a common compound of phthalates and contaminant in food/feed, is teratogenic in animals (Lyche J L. et al., (2009), Reproductive and developmental toxicity of phthalates. J. Toxicol. Environ. Health B Crit Rev. 12 (4) 225-249). Moreover, there are reports confirming that DBP can cause childhood diseases, i.e., premature breast development, asthma and allergic symptoms etc. (Braun J M., (2013), Phthalate exposure and children's health. Curr Opin Pediatr 2 (2) 247-254).

Nevertheless, one of the main routes of exposure is through the ingestion of chemically contaminated food. These substances are classified as Endocrine Disrupting Chemicals (EDCs), interfere with the body's endocrine system inducing developmental and reproductive toxicity, which has been extensively reviewed and reported by the National Toxicology Program—Center for the Evaluation of Risks to Human Reproduction Program. Additionally, phthalates and bisphenol A have been associated with various adverse effects related to cardiovascular diseases, cancer, neurotoxicity, asthma and allergies, insulin resistance and type II diabetes, overweight and obesity.

Studies on experimental animals have demonstrated that individual phthalates with a similar mechanism of action (DEHP and DBP), but different active metabolites (monoethylhexyl phthalate (MEHP) versus monobutyl phthalate (MBP)), can elicit dose-additive effects when administered as a mixture (Howdeshell et al., (2008), Mechanisms of action of phthalate esters, individually and in combination, to induce abnormal reproductive development in male laboratory rats. Environ Res. 108 (2) 168-176). Similar dose additive effects have been reported for mixtures of phthalates with antiandrogenic pesticides of differing mechanisms of action. In a subchronic toxicity study on rats, Zhang et al. (2013), (Combined subchronic toxicity of bisphenol A and dibutyl phthalate on male rats. Biomed Environ Sci. 26 (1) 63-69) have shown that, under combined DBP and BPA treatment, the expression levels of sex hormone genes were higher compared to the control group, suggesting an additive or a synergistic effect (Zhang et al., (2019), Association between exposure to a mixture of phenols, pesticides and phthalates and obesity: Comparison of three statistical models. Environ Int 123 325-336). Previous results have demonstrated that the mixture of DEHP, DBP and BPA produced more pronounced changes in lipid profile, liver-related biochemical and hormonal parameters, as well as glucose level of rats exposed for 28 days in comparison with the changes induced by single substances (Baralić et al., (2020), Toxic effects of the mixture of phthalates and Bisphenol A-Subacute oral toxicity study in Wistar rats Int J Environ Res Public Health 17 (3) 746).

### Removal of Toxins and Bioremediation.

All organisms, humans, animals, plants and bacteria are exposed to many toxic and potentially deleterious compounds that are found in the environment, food chain, water supply and soil. According to a comprehensive, public WHO study on diseases due to unhealthy environments nearly 25% of the global disease burden could be prevented by reducing environmental risks and confirms that eliminating hazards and reducing environmental risks would greatly benefit our health and wellbeing.

Bioremediation is a branch of biotechnology that employs the use of living organisms, like plants, fungi and bacteria, in the removal of contaminants, pollutants, and toxins from soil, and water. Bioremediation may be used to clean up contaminated groundwater or environmental problems, such as oil spills, by degrading, removing, changing, immobilizing, or detoxifying various chemicals and physical pollutants from the environment. Plants, fungi as well as bacteria have been investigated for their use in sequestration and detoxification of pollutants like heavy metals and have shown success (Singh, J. S., Abhilash, P. C., Singh, H. B., Singh, R. P., & Singh, D. P. (2011). Genetically engineered bacteria: an emerging tool for environmental remediation and future research perspectives. Gene, 480 (1-2), 1-9; Rajkumar, M., Ae, N., Prasad, M. N. V., & Freitas, H. (2010). Potential of siderophore-producing bacteria for improving heavy metal phytoextraction. Trends in biotechnology, 28 (3), 142-149).

The use of microbial metabolic potential for eliminating environmental pollutants provides a safe and economic alternative to their disposal in waste dump sites and to commonly used physico-chemical strategies. Microorganisms capable of mineralizing a variety of toxic compounds under laboratory conditions have been isolated. The accumulation in the environment of highly toxic and persistent compounds, however, emphasizes the fact that the natural metabolic diversity of the microbes is often insufficient to protect the biosphere from anthropogenic pollution. Many recalcitrant chemicals contain structural elements or substituents that do not (or seldom) occur in nature (xenobiotics). Presumably, because of the novelty of these compounds, microorganisms have not evolved appropriate metabolic pathways capable of degrading them. Whereas for some xenobiotics no degradative routes have been described, others are transformed incompletely or inefficiently, or the complex mixtures of contaminants prevent degradation by existing pathways (Pieper, D. H., & Reineke, W. (2000). Engineering bacteria for bioremediation. Current opinion in biotechnology, 11 (3), 262-270).

Several bacteria degrading herbicides in soil including glyphosate have been investigated (Cullington, J. E., & Walker, A. (1999). Rapid biodegradation of diuron and other phenylurea herbicides by a soil bacterium. Soil Biology and Biochemistry, 31 (5), 677-686; Zabaloy, M. C., Garland, J. L., & Gomez, M. A. (2010). Assessment of the impact of 2, 4-dichlorophenoxyacetic acid (2, 4-D) on indigenous herbicide-degrading bacteria and microbial community function in an agricultural soil. Applied Soil Ecology, 46 (2), 240-246; Hernández García, M., Morgante, V., Ávila Perez, M., Villalobos Biaggini, P., Miralles Noé, P., González Vergara, M., & Seeger Pfeiffer, M. (2008). Novel s-triazine-degrading bacteria isolated from agricultural soils of central Chile for herbicide bioremediation. Electronic Journal of Biotechnology, 11 (5), 5-6; Zhan, H., Feng, Y., Fan, X., & Chen, S. (2018). Recent advances in glyphosate biodegradation. Applied microbiology and biotechnology, 102 (12), 5033-5043). These studies identified Stenotrophomonas spp., Arthrobacter spp., and Streptomyces spp. as encoding such activities, however no study isolated and characterized degrading strains at high resolution.

Bioremediation methods, however, are very limited because any environmental cleanups of agricultural toxins are very difficult to implement on a scale matching the vast areas of glyphosate usage. Additionally, the organisms described for such potential remediation are soil bacteria and can unfortunately not be a priori safely used by exposed humans or animals. Health-conscious consumers have therefore become interested in food that is able to protect from the effects of environmental toxins, and a fast-growing over-the-counter market has arisen under the guise of ‘detox’ food. Most of the so-called “detox” products have unfortunately little rationale or clinical evidence to support their use, and few proven and effective products exist despite a growing need.

The use of well-studied food microorganisms, however, shows promising results through the administration of probiotics. It has found much interest amongst consumers, as well as healthcare and research professionals. Probiotics can be defined as nonpathogenic food-grade microorganisms that, when ingested, exert a positive influence on the health or physiology of the host (Fuller, R. (1989). Probiotic in man and animals. J. Appl. Bacteriol., 66, 131-139).

They consist of either yeast or bacteria, especially lactic acid bacteria (LAB). Their fate in the environment that they are applied to, for example the gastrointestinal tract and their effects differ strongly among strains even within a particular species (Marteau, P., Pochart, P., Bouhnik, Y., & Rambaud, J. C. (1993). The fate and effects of transiting, nonpathogenic microorganisms in the human intestine. Intestinal Flora, Immunity, Nutrition and Health, 74, 1-21).

The mechanisms of action of probiotics are still a matter of research but are believed to be through modulation of the endogenous microbiome, the blocking of the attachment of deleterious bacteria, or of the immune system (Marteau, P., Pochart, P., Bouhnik, Y., & Rambaud, J. C. (1993). The fate and effects of transiting, nonpathogenic microorganisms in the human intestine. Intestinal Flora, Immunity, Nutrition and Health, 74, 1-21).

Probiotic Lactobacilli and Bifidobacteria have been shown to help manage several gut pathologies. For example, U.S. Pat. No. 6,641,808 disclosing the use of Lactobacilli for the treatment of obesity; U.S. Pat. No. 5,531,988, discloses a mixture of an immunoglobulin and a bacterium, such as Lactobacillus or Bifidobacterium or mixtures thereof, that may be used to treat diarrhea, constipation, and gas/cramps; U.S. Pat. No. 6,080,401 discloses a combination of probiotics having Lactobacillus acidophilus and Bifidobacterium bifidus and herbal preparations for aiding in weight loss, and so forth.

The ability of probiotic products to ameliorate toxins has been studied to a much lesser degree and has focused mainly on binding of metals or mycotoxins like aflatoxin (Hernandez-Mendoza, A., Garcia, H. S., & Steele, J. L. (2009). Screening of Lactobacillus casei strains for their ability to bind aflatoxin B1. Food and chemical toxicology, 47 (6), 1064-1068); and detoxify or bind and negate other mycotoxins, e.g. with Bacillus animalis (Fuchs, S., Sontag, G., Stidl, R., Ehrlich, V., Kundi, M., & Knasmüller, S. (2008). Detoxification of patulin and ochratoxin A, two abundant mycotoxins, by lactic acid bacteria. Food and chemical toxicology, 46 (4), 1398-1407). The use of food-grade bacteria to degrade certain agricultural chemicals like atrazine has been explored. Equally non-food grade soil bacteria that degrade glyphosate have been described (Wang, G. H., Berdy, B. M., Velasquez, O., Jovanovic, N., Alkhalifa, S., Minbiole, K. P., & Brucker, R. M. (2020). Changes in microbiome confer multigenerational host resistance after sub-toxic pesticide exposure. Cell host & microbe, 27 (2), 213-224; Zhu, J., Zhao, Y., Li, X., Chen, W., & Huang, R. (2018). Study on the characteristics and utilization of a triclopyr butoxyethyl ester degrading bacterium, Lactobacillus buchneri TBE-6. Fresenius Environmental Bulletin, 27 (5), 3156-3161; Li, C., Ma, Y., Mi, Z., Huo, R., Zhou, T., Hai, H., & Zhang, H. (2018). Screening for Lactobacillus plantarum strains that possess organophosphorus pesticide-degrading activity and metabolomic analysis of phorate degradation. Frontiers in microbiology, 9, 2048; Manogaran, M., Shukor, M. Y., Yasid, N. A., Johari, W. L. W., & Ahmad, S. A. (2017). Isolation and characterisation of glyphosate-degrading bacteria isolated from local soils in Malaysia. Rendiconti Lincei, 28 (3), 471-479; Singh, B., & Singh, K. (2016). Microbial degradation of herbicides. Critical reviews in microbiology, 42 (2), 245-261; Huang, X., He, J., Yan, X., Hong, Q., Chen, K., He, Q., . . . & Jiang, J. (2017). Microbial catabolism of chemical herbicides: microbial resources, metabolic pathways and catabolic genes. Pesticide biochemistry and physiology, 143, 272-297).

In view of the possible problems associated with the widespread exposure to glyphosate, and the problems associated with an environmental cleanup of vast areas, it would be advantageous to provide food grade bacteria that can sequester, bind, remove, inactivate, or degrade glyphosate derived from the environment via water, plants, feed and food within or outside the gastrointestinal tract, or cause the subject organism to sequester, bind, remove, inactivate, or degrade it. The present invention meets these, and other needs as detailed below.

### SUMMARY OF THE INVENTION

It is an object of the present teachings to provide for food-grade bacteria, e.g. probiotics, or extracts thereof for the removal and/or neutralization of glyphosate, phthalates, BPA, and other toxic compounds from an environment or from a substance to which the food-grade bacteria are exposed to, that solve the deficiencies inherent in other potential detoxification treatments.

The present disclosure provides methods and uses of food grade bacteria, e.g. probiotics, for removal and/or neutralization of toxic compounds, such as glyphosate, phthalates, and BPA, found in the internal environment of animals and humans, in the environment to which the animals or humans are exposed to or in a substance ingested or to be ingested that may avoid adverse side effects, is reasonable in cost, and may be beneficial in reducing the risk of damages by glyphosate, phthalates, and BPA. Further, the present invention is relatively easy to manufacture and deliver to a subject. It is an object of the present invention to provide for food grade bacteria, or extracts thereof, to detoxify and/or sequester glyphosate, phthalates, and BPA.

The present disclosure can remove various toxic compounds from an environment, including living subjects. The toxic compounds can be selected from the group consisting of glyphosate, phthalates, BPA, and other known pesticides, herbicides and derivatives.

As such, in one embodiment, the present invention provides food-grade bacteria, e.g. probiotics, or extracts thereof for removing glyphosate, phthalates, and BPA from a substance, a mixture of substances or environment to which the food-grade bacteria is exposed.

In one embodiment, the present invention provides for a composition comprising bacteria listed in Table 1 and a suitable carrier, whereby the composition comprises an effective dose of the food-grade bacteria to remove glyphosate, phthalates, and BPA from a substance, mixture of substances, or environment to which the food-grade bacteria are exposed.

In one embodiment of the composition of the present invention, the effective dose is at least about 1×10⁸ of the food-grade bacteria per milliliter or gram of the suitable carrier.

In another embodiment of the composition of the present invention, the suitable carrier is a water and carbohydrate-containing medium.

In another embodiment of the composition of the present invention, the carbohydrate-containing medium is a milk, or milk protein-based product such as organic yogurt.

In another embodiment of the composition of the present invention, the carbohydrate-containing medium is selected from organic soft drinks, juices, kombucha, cider, kefir, plant-based milks (soy, oatmeal, coconut. cashew, macadamia).

In another embodiment of the composition of the present invention, the carbohydrate-containing medium is selected from confectionary products such as, chocolate, hot chocolate powder.

In another embodiment of the composition of the present invention, the carbohydrate-containing medium is selected from organic breads and cereals.

In another embodiment of the composition of the present invention, the carbohydrate-containing medium is selected from protein powder drinks and protein bars.

In another embodiment of the composition of the present invention, the carbohydrate-containing medium is selected from organic gummies, and fruit roll up snacks.

In another embodiment of the composition of the present invention, the toxic compound is selected from the group consisting of glyphosate, phthalates, BPA and commercially used formulations thereof.

In another embodiment of the composition of the present invention, the toxic compound is selected from the group consisting of glyphosate, phthalates, BPA, and other known pesticides, herbicides and derivatives.

In another embodiment of the composition of the present invention, the food-grade bacteria are provided dead or alive.

In another embodiment of the composition of the present invention, the food-grade bacteria are provided as an extract.

In another embodiment of the composition of the present invention, the composition comprises a combination of two or more different species of food-grade, or feed-grade bacteria.

In another embodiment of the composition of the present invention, the composition comprises a combination of two or more strains from the group of food-grade bacteria listed in Table 1 shown below, alone or in combination with other food-grade, non-glyphosate and phthalates degrading bacteria.

In another embodiment, the present invention is a composition, the composition including food-grade bacteria, a carrier, and an animal's feed, such as pet foods, pet chews, or powdered additives etc., wherein the-food-grade bacteria is capable of removing glyphosate and phthalates from a substance or environment to which the food-grade bacteria is exposed and the food-grade bacteria comprises a bacterial isolate selected from the group consisting of the food-grade bacteria listed in Table 1 or any combination thereof.

In one embodiment, the present invention is a method for reducing a subject uptake of glyphosate, phthalates, and BPA consumed by the subject, the method including administering to the subject an effective dose of a food-grade bacteria composition capable of sequestering glyphosate and phthalates consumed by the subject.

In another embodiment, the present invention is a method for removing glyphosate, phthalates, and BPA from a substance or environment which is contaminated or suspected of being contaminated with the toxic compound is provided, the method including contacting the substance or environment with food-grade bacteria from Table 1 capable of removing the toxic compound from the substance or the environment.

In one embodiment, the present invention is a method of reducing the toxic effects of glyphosate, phthalates, and BPA in a subject, the method including: administering to the subject a therapeutically effective amount of a food-grade bacteria capable of removing glyphosate from the subject. In yet another embodiment of the method of the present invention, in addition to glyphosate, phthalates, and BPA, and other known pesticides, herbicides and derivatives can be removed.

In one embodiment, the present invention is a method for obtaining a cell fraction capable of removing glyphosate, phthalates, and BPA from an environment or subject, including the steps of: a) culturing a strain of bacteria from Table 1, and b) recovering the cell fraction capable of removing glyphosate, phthalates, and BPA from an environment or subject from the culture in step a).

In another embodiment of the use of the food-grade bacteria from Table 1, the food-grade bacteria are provided as an extract.

In another embodiment of the use of the food-grade bacteria, the food-grade bacteria are provided as one or more strains of bacteria listed in Table 1.

In another embodiment of the use of the food-grade bacteria from Table 1, the bacteria are provided as a combination with one or more species of additional food-grade organisms not listed in Table 1.

In another embodiment, the present invention provides for a method for removing glyphosate, phthalates, and BPA from a substance which is suspected of being contaminated with glyphosate and phthalates comprising contacting the substance with food-grade bacteria from Table 1 or an extract thereof. In yet another embodiment of the method of the present invention, in addition to removing glyphosate, phthalates, and BPA, and other known pesticides, herbicides and derivatives can be removed.

### BRIEF DESCRIPTION OF THE FIGURES

FIG. 1 is a bar graph of the relative quantities as a percentage of glyphosate found remaining in growth medium after cultivation with the probiotic combinations A, B, C, and D of the invention.

FIG. 2 is a is a bar graph of the relative quantities of glyphosate found remaining in growth medium after cultivation with various probiotic strains in the probiotic combinations of the invention, showing adsorption of glyphosate on the cell surface of bacterial strains.

FIG. 3 is a bar graph of the relative quantities of glyphosate (ng/g of stool) as measured in stool of mice in 4 groups, showing probiotic mix treated mice retained higher levels of glyphosate in stool (not absorbed) compared to control mice (Group 1).

FIG. 4 is a bar graph of the relative quantities of glyphosate (ng/ml of serum) in serum showing mice receiving higher doses of probiotic compositions of the invention showed a 46% reduction in serum glyphosate (Group 3) compared to control mice.

FIG. 5 is a bar graph of the relative quantities of glyphosate (ng/ml of liver) as measured in the liver of mice of Group 4 fed probiotic compositions of the invention showing a 20% reduction in glyphosate retention versus control group 1.

FIG. 6 is a bar graph of BPA binding as measured by GC-MS testing of BPA binding activity comparing control medium (dark bars) set to 100% BPA for comparison to BPA remaining in cultures (light bars) containing L. salivarius (L.s.), L. plantarum (L.p.) and B. coagulans (B.c.).

FIG. 7 is a bar graph of BPA binding in heat-treated cells as measured by GC-MS of average BPA measurements from triplicate cultures of L. salivarius (L.s.), L. plantarum (L.p.) and B. coagulans (B.c.).

### DETAILED DESCRIPTION

Technical and scientific terms used herein have the same meaning as commonly understood by one of ordinary skill in the art to which this invention belongs. Also, unless indicated otherwise, except within the claims, the use of “or” includes “and” and vice versa. Non-limiting terms are not to be construed as limiting unless expressly stated or the context clearly indicates otherwise (for example “including”, “having” and “comprising” typically indicate “including without limitation”). Singular forms including in the claims such as “a”, “an” and “the” include the plural reference unless expressly stated otherwise.

The expression “food-grade bacteria” refers to any bacteria, alive or dead, that have no harmful effect on human health or that have a GRAS (generally recognized as safe) status, or a similar designation in European or other jurisdictions. Non-limiting examples of food-grade bacteria particularly suitable for the purpose of the present invention are listed in Table 1. The term “probiotic” as used in this document refers to food-grade bacteria which perform beneficial functions to subject organisms when they are present and alive in viable form in the subject organisms.

“Food production animal” is used herein to describe any animal that is involved and used for human or animal consumption. A food production animal can be, but not limited to, a ruminant animal such as beef and dairy cattle, pigs, lamb, chicken, turkey or any other fowl, or aquatic animals including shrimp, lobster or fish used for human or other animal consumption, as well as insects used as feed or food, or insects used to make food e.g. honey bees.

As used herein, the term “removing a toxic compound from a substance or environment” refers to a removal of glyphosate or more toxic compounds that can be tested as described in at least one of the examples below.

“Subject” or “subjects” are used herein to describe a member of the animal kingdom, including food production animals and humans.

The present invention also encompasses mutant strains derived from a parent strain. These mutant strains can be strains wherein one or more endogenous gene(s) of the parent strain has (have) been mutated, for instance to modify some of its metabolic properties (e.g., its ability to ferment sugars, its resistance to acidity, its survival to transport in the gastrointestinal tract, its post-acidification properties or its metabolite production, or the degree of its ability to degrade glyphosate). These strains can be obtained from a microbial strain by means of the conventional techniques for random natural or induced mutagenesis, in vivo or laboratory adaptive evolution, genetic screens, or by high throughput screening methods.

The present invention also encompasses cell fractions which can be obtained from the listed bacterial strains. They may also be culture supernatants or fractions of these supernatants.

The present invention also encompasses a method for obtaining such glyphosate removing cell fraction, comprising the steps of:
a) culturing a strain listed in Table 1, and
b) obtaining and/or recovering the cell fraction from the culture in step a).

In compositions of the invention, said strains can be used in the form of whole bacteria which may be living or dead. Alternatively, said strain can be used in the form of a bacterial lysate or in the form of bacterial fractions; the bacterial fractions suitable for this use can be chosen, for example, by testing their properties on glyphosate removal from an aqueous environment. Preferably the bacterial cells are present as living, viable cells.

### Food-Grade Bacteria for Removing Glyphosate

In one embodiment, the present invention relates to food-grade microorganisms or extracts thereof, including probiotics, capable of removing or sequestering glyphosate from an environment to which the food-grade bacteria are exposed, or from a substance or a subject which may have or may be suspected of containing glyphosate. Substances may include edible compositions, such as plant-based or animal-based foods or feeds, and may also include drinkable solutions, including water, milk, extracts and other beverages. Substances may also include raw agricultural products used to produce foods and drinkable solutions. As such, the present invention relates also to methods of using the food-grade bacteria of the present invention to prevent the uptake of glyphosate by a subject, or in methods to filter glyphosate out of substances prior to exposing a subject to said substances, or to remove glyphosate after subjects are being exposed. The environment may include an aqueous environment, such as the gastrointestinal tract of a subject, or the environment in which the subject or its feed or food resides, such as a pond, beehive, feed or food storage or other environment.

The food grade bacteria may be any type of bacteria, yeasts or algae that may be capable of removing glyphosate from subjects, foods or solutions that may be consumed by a subject (animal or human), or from ingredients used in the manufacture of said foods or solutions. Table 1 includes food-grade bacteria that may be used with the present invention. In a preferred aspect, the food-grade bacteria may be aerobically, semi-aerobically or anaerobically grown and may be selected from the group consisting of the food-grade bacteria of Table 1. Administration of the food-grade bacteria, or extract thereof, to a subject may be accomplished by any method likely to introduce the organisms into the gastro-intestinal tract of the subject. The bacteria can be mixed with a carrier and applied to liquid or solid feed or to drinking water. The carrier material should be non-toxic to the subject. When dealing with live food-grade bacteria, the carrier material should also be non-toxic to the food-grade bacteria. When dealing with live food-grade bacteria the carrier, preferably, may include an ingredient that promotes viability of the bacteria during storage. The food-grade bacteria may also be formulated as a paste to be directly injected into a subject's mouth. The formulation may include added ingredients to improve palatability, improve shelf-life, impart nutritional benefits, and the like. If a reproducible dose is desired, the food-grade bacteria can be administered by any measuring device. The amount of food-grade bacteria to be administered is governed by factors affecting efficacy. When administered in food, feed or drinking water the dosage can be spread over a short time, or a period of days or even weeks. The cumulative effect of lower doses administered over several days may be greater than a single larger dose thereof. One or more strains of food-grade bacteria may be administered together. A combination of strains may be advantageous because subjects may differ as to the strain which is most persistent in a given species or individual.

The present invention is also directed to extracts or fragments of food-grade bacteria that may be capable of removing or sequestering glyphosate from a substance or sample.

### Applications

Food-grade bacteria of the present invention may be used as a preventive measure, to reduce the exposure of a subject to glyphosate, phthalates, or plasticizers for example bisphenol A (BPA) from consumables or environments where the compound is present. Food grade bacteria of the present invention may also be used to substantially reduce glyphosate from a subject by administering the food-grade bacteria, or extracts thereof, to the subject carrying the toxic compound.

The methods for administering food-grade bacteria may essentially be the same, whether for prevention or treatment. By routinely administering an effective dose to a subject, the risk of contamination by the undesired toxin may be substantially reduced or substantially eliminated by a combination of prevention and treatment.

The food-grade bacteria may also be used, according to another embodiment of the present invention, to food producing animals, like aquatic animals such as fish and shrimp, or insects like honey bees, or insects used as edible food or feed. In one embodiment, food-grade bacteria of the present invention may, for example, be added to tanks and ponds containing aquatic animals, or to water or feed materials in insect rearing facilities or bee hives.

### Preparation and Administration

Although this invention is not intended to be limited to any particular mode of application, oral administration of the compositions is preferred. One food-grade bacterium spp. may be administered alone or in conjunction with a second, different food-grade bacterium spp. Any number of different food-grade bacteria may be used in conjunction. By “in conjunction with” is meant together, substantially simultaneously or sequentially. The compositions may be administered in the form of tablet, pill, capsule, paste, powder or liquid, for example. One preferred form of application involves the preparation of an encapsulated, freeze-dried material comprising the composition of the present invention. Another preferred form of application involves the preparation of a lyophilized capsule of the present invention. Still another preferred form of application involves the preparation of a heat dried capsule of the present invention.

By “amount effective” as used herein is meant an amount of food-grade bacterium or bacteria listed in Table 1, e.g., Lactobacillus salivarius, Bacillus coagulans, Lactobacillus plantarum, high enough to significantly and positively modify the condition to be treated but low enough to avoid any serious side effects (at a reasonable benefit/risk ratio), within the scope of sound judgment. An effective amount of said bacteria will vary with the particular goal to be achieved, the age and physical condition of the subject being treated, the duration of treatment, and the specific bacteria employed. The effective amount of Lactobacillus salivarius, Lactobacillus plantarum, or Bacillus coagulans will thus be the minimum amount which will provide the desired removal of glyphosate.

A significant practical advantage is that the food-grade bacteria, e.g. Lactobacillus salivarius, Lactobacillus plantarum, Bacillus coagulans, may be administered in a convenient manner such as by the oral, or suppository routes. Depending on the route of administration, the active ingredients which comprise food-grade bacteria may be required to be coated in a material to protect said organisms from the action of enzymes, acids and other natural conditions which may inactivate said organisms. In order to administer food-grade bacteria they may be coated by, or administered with, a material to prevent inactivation. For example, food-grade bacteria may be co-administered with enzyme inhibitors or in liposomes. Enzyme inhibitors include pancreatic trypsin inhibitor, diisopropylfluorophosphate (DFP) and trasylol. Liposomes include water-in-oil-in-water P40 emulsions as well as conventional and specifically designed liposomes which transport bacteria or their byproducts to an internal target of a host subject. Dispersions can also be prepared, for example, in glycerol, liquid polyethylene glycols, and mixtures thereof, and in oils.

In the case of sterile powders for the preparation of sterile solutions, the preferred methods of preparation are vacuum-drying and the freeze-drying technique which yield a powder of the active ingredient plus any additional desired ingredient from previously sterile-filtered solution thereof. Additional preferred methods of preparation include but are not limited to lyophilization and heat-drying.

When the food-grade bacteria are suitably protected as described above, the active compound may be orally administered, for example, with a diluent or with an edible carrier, or it may be enclosed in hard or soft shell gelatin capsule, or it may be compressed into tablets designed to pass through the stomach (i.e., enteric coated), or it may be incorporated directly with the food or feed of dietary preparations. For oral therapeutic administration, the food-grade bacteria may be incorporated with excipients and used in the form of ingestible tablets, buccal tablets, troches, capsules, elixirs, suspensions, syrups, wafers, and the like.

The tablets, troches, pills, capsules, and the like, as described above, may also contain the following: a binder such as gum tragacanth, acacia, corn starch or gelatin; excipients such as dicalcium phosphate; a disintegrating agent such as corn starch, potato starch, alginic acid, and the like; a lubricant such as magnesium stearate; and a sweetening agent such as sucrose, lactose or saccharin may be added or a flavoring agent such as peppermint, oil of wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills or capsules or Lactobacillus salivarius, Lactobacillus plantarum, Bacillus coagulans in suspension may be coated with shellac, food glace, sugar or both. A syrup or elixir may contain the active compound, sucrose or sugar replacements as a sweetening agent, methyl and propylparabens as preservatives, a dye and flavoring such as cherry or orange flavor. Of course, any material used in preparing any dosage unit form should be pure and substantially non-toxic in the amounts employed. In addition, the food-grade organism may be incorporated into sustained-release preparations and formulations.

The specification for the dosage unit forms of the invention may be dictated by and may be directly depending on (a) the unique characteristics of the particular food-grade bacteria and the particular effect to be achieved, and (b) the limitations inherent in the art of compounding such food-grade bacteria for the establishment and maintenance of a healthy flora in the intestinal tract.

The food-grade organism is compounded for convenient and effective administration in effective amounts with a suitable food acceptable carrier in dosage unit form as herein disclosed. A unit dosage form can, for example, contain the principal active compound in an amount approximating 109 viable or non-viable, e.g., Lactobacillus salivarius, Lactobacillus plantarum, Bacillus coagulans per ml. In the case of compositions containing supplementary ingredients such as prebiotics, the dosages are determined by reference to the usual dose and manner of administration of the said ingredients.

The pharmaceutically acceptable carrier may be in the form of milk or portions thereof including yogurt, skim milk, skim milk powder. Non-milk or non-lactose containing milk replacing products based on soy, oats, rice, almonds or the like may also be employed. The skim milk powder or above-mentioned replacements are conventionally suspended in phosphate buffered saline (PBS), autoclaved or filtered to eradicate proteinaceous and living contaminants, then freeze dried, heat dried, vacuum dried, or lyophilized.

Some other examples of substances which can serve as carriers are sugars, such as lactose, glucose and sucrose; starches such as corn starch and potato starch; cellulose and its derivatives such as sodium carboxymethycellulose, ethylcellulose and cellulose acetates; powdered tragacanth; malt; gelatin; talc; stearic acids; magnesium stearate; calcium sulfate; calcium carbonate; vegetable oils, such as peanut oils, cotton seed oil, sesame oil, olive oil, com oil and oil of theobroma; polyols such as propylene glycol, glycerin, sorbitol, mannitol, and polyethylene glycol; agar; alginic acids; pyrogen-free water; isotonic saline; cranberry, extracts and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, antioxidants and preservatives, can also be present.

Accordingly, the subject may be orally administered an effective amount of at least one food-grade bacteria and an acceptable carrier in accordance with the present invention. The food-grade bacteria may be selected from the group comprising the bacteria listed in Table 1.

TABLE 1
Microorganisms removing glyphosate.

| Genus | Species | Strain |
|---|---|---|
| Lactobacillus | salivarius | SD-5208 |
| Bacillus | coagulans | NTCC 5856 |
| Lactobacillus | plantarum | HA-119 |

The foregoing disclosure generally describes the present invention. Changes in form and substitution of equivalents are contemplated as circumstances may suggest or render expedient. Although specific terms have been employed herein, such terms are intended in a descriptive sense and not for purposes of limitation.

### EXAMPLES

The examples are described for the purposes of illustration and are not intended to limit the scope of the invention.

### Example 1

Example 1—Demonstration of removal of glyphosate by Lactobacillus salivarius SD-5208 from an aqueous environment.

Filter sterilized MRS liquid medium (per liter; 10 g casein peptone, tryptic digest, 10 g beef extract, 5 g yeast extract, 20 g glucose, 1 g Tween 80, 2 g K₂HPO₄, 2 g ammonium citrate, 5 g sodium acetate, 100 mg magnesium sulphate, 50 mg manganese sulphate, pH adjusted by 6.5) is inoculated with a multi-strain probiotic (Table 2), supplemented with 1 mM glyphosate, and is incubated overnight (14 hrs.) at 37° C. with shaking. The cultures are then transferred to a sterile microcentrifuge tube and cells are pelleted at 13,000 rpm for 3 minutes. An aliquot of the liquid medium is removed for analysis of the glyphosate concentration by GC-MS. GC-MS is conducted using standard methods (Bunk, B., Kucklick, M., Jonas, R., Münch, R., Schobert, M., Jahn, D., & Hiller, K. (2006). MetaQuant: a tool for the automatic quantification of GC/MS-based metabolome data. Bioinformatics, 22 (23), 2962-2965) and described briefly here.

Gas Chromatography-Mass Spectrometry (GC-MS) Sample Preparation and Analysis. Medium samples (40 μl) are mixed with 10 μl 1 mM L-norvaline (internal standard) and dried under vacuum using a Speedvac® centrifugal evaporator. At the same time, separate tubes containing varying amounts of standards (sarcosine, aminomethyl-phosphonic acid, cyanuric acid, glyphosate and N-acetyl-glyphosate) are evaporated. Dried samples and standards are derivatized with the addition of 60 μl of a 1:1 mix of N-tertbutyldimethylsilyl-N-methyltrifluoroacetamide (MTBSTFA) (Soltec Ventures) and pyridine and incubated for 60 min at 80° C. The derivatized samples and standards are analyzed by GC-MS using an Rxi-5 ms column (15 m×0.25 i.d.×0.25 μm, Restek) installed in a Shimadzu QP-2010 Plus gas chromatograph-mass spectrometer (GC-MS). The GC-MS is programmed with an injection temperature of 250° C., 1.0 μl injection volume and split ratio 1/10. The GC oven temperature is initially 130° C. for 4 min, rising to 250° C. at 6° C./min, and to 280° C. at 60° C./min with a final hold at this temperature for 2 min. GC flow rate, with helium as the carrier gas, is 50 cm/s. The GC-MS interface temperature is 300° C. and (electron impact) ion source temperature is 200° C., with 70 eV ionization voltage. Data from standards are used to construct calibration curves in MetaQuant (Bunk et al., 2006 supra). Metabolite amounts in samples are determined from these calibrations and corrected for recovery of the internal standard. Chemical standards (glyphosate) are used to determine elution time and for quantification of experimental samples.

TABLE 2
List of bacterial species contained in Probiotic Combination A.

| Genus | Species | Strain |
|---|---|---|
| Lactobacillus | salivarius | SD-5208 |
| Lactobacillus | acidophilus | SD-5219 |
| Lactobacillus | casei | SD-5213 |
| Lactobacillus | plantarum | SD-5209 |
| Lactobacillus | rhamnosus | SD-5217 |
| Lactobacillus | brevis | SD-5214 |
| Lactobacillus | bulgaricus | SD-6833 |
| Lactobacillus | gasseri | SD-5585 |
| Lactococcus | lactis | SD-5584 |
| Bifidobacterium | longum | SD-5588 |
| Bifidobacterium | bifidum | SD-6576 |
| Bifidobacterium | infantis | SD-6720 |
| Streptococcus. | thermophilus | SD-5207 |
| Bifidobacterium | lactis | SD-5219 |

The probiotic mixture results in removal of 67% of input glyphosate compared to control media without bacteria.

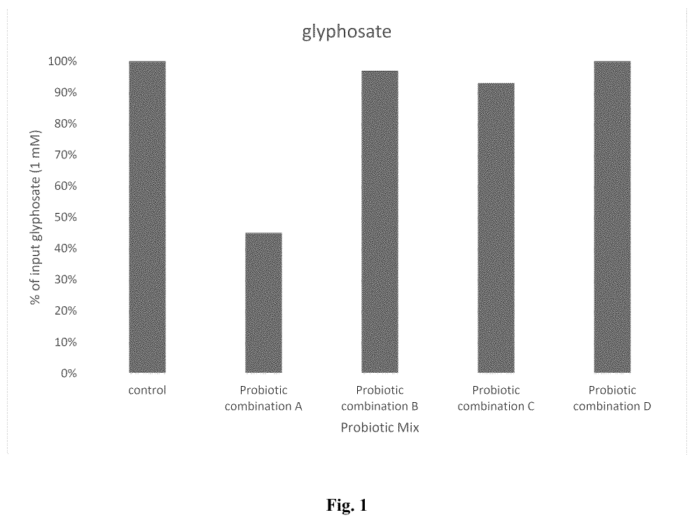

To confirm the activity and identity of the active bacterium, 25 colony purified clones are generated by plating liquid cultures on solid MRS media to create 5 pools containing 5 clones/pool that are inoculated into MRS media supplemented with 1 mM glyphosate and incubated overnight (12 hrs.) at 37° C. with shaking. The cultures are then transferred to a sterile microcentrifuge tube and cells are pelleted at 13,000 rpm for 3 minutes. The supernatants are then rescreened to GC-MS to identify the pool with the best glyphosate degradation potential (see FIG. 1). Pool 5, containing clones 21-25 is then deconvoluted further by repeating liquid cultures for individual clones in MRS media supplemented with 1 mM glyphosate as described above. The cleared supernatants are subjected to GC-MS and clones 21, 23 and 25 are used for genomic DNA isolation, using standard commercial kits (Qiagen). The recovered genomic DNAs are used as a template to amplify the 16S rDNA gene. The resulting amplicons are purified and subjected to DNA sequencing using the forward primer used for PCR. The resulting sequences are used to conduct BLAST searches against the NCBI database. All 3 clones revealed a best match to Lactobacillus salivarius, consistent with the presence of the said bacterium in the original probiotic mixture, corresponding to strain SD-5208.

TABLE 3

| | +L. salivarius | |
|---|---|---|
| Growth Medium | SD-5208 | −L. salivarius |
| Remaining glyphosate Concentration | 25% | 100% |
| (of 1 mM glyphosate) | | |

As illustrated in Table 3, 75% of glyphosate is removed from solution in the presence of Lactobacillus salivarius SD-5208, while no glyphosate is removed in the control not containing bacteria. Glyphosate removal is deemed significant (p<0.05) by an ANOVA one-way analysis of variance.

### Example 2

Example 2—Demonstration of removal of glyphosate by Lactobacillus plantarum HA-119 from an aqueous environment.

Filter sterilized MRS liquid medium (per liter; 10 g casein peptone, tryptic digest, 10 g beef extract, 5 g yeast extract, 20 g glucose, 1 g Tween 80, 2 g K₂HPO₄, 2 g ammonium citrate, 5 g sodium acetate, 100 mg magnesium sulphate, 50 mg manganese sulphate, pH adjusted by 6.5) is inoculated with a multi-strain probiotic (Table 4), supplemented with 1 mM glyphosate, and is incubated overnight (14 hrs.) at 37° C. with shaking. The cultures are then transferred to a sterile microcentrifuge tube and cells are pelleted at 13,000 rpm for 3 minutes. An aliquot of the liquid medium is removed for analysis of the glyphosate concentration by GC-MS.

TABLE 4
List of bacterial species contained in Probiotic Combination.

| Genus | Species | Strain |
|---|---|---|
| Lactobacillus | salivarius | HA-118 |
| Lactobacillus | acidophilus | HA-122 |
| Lactobacillus | casei | HA-108 |
| Lactobacillus | plantarum | HA-119 |
| Lactobacillus | rhamnosus | HA-111 |
| Lactobacillus | rhamnosus | HA-114 |
| Bifidobacterium | breve | HA-129 |
| Bifidobacterium | longum | HA-135 |
| Bifidobacterium | bifidum | HA-132 |
| Lactobacillus | fermentum | HA-179 |
| Bifidobacterium | lactis | HA-194 |
| Lactobacillus | paracasei | HA-196 |

The probiotic mixture results in removal of 46% of input glyphosate compared to control media without bacteria.

To confirm the activity and identity of the active bacterium, 25 colony purified clones are generated by plating liquid cultures on solid MRS media to create 5 pools containing 5 clones/pool that are inoculated into MRS media supplemented with 1 mM glyphosate and incubated overnight (12 hrs.) at 37° C. with shaking. The cultures are then transferred to a sterile microcentrifuge tube and cells are pelleted at 13,000 rpm for 3 minutes. The supernatants are then rescreened to GC-MS to identify the pool with the best glyphosate degradation potential. The pool displaying the best glyphosate removal is then deconvoluted further by repeating liquid cultures for individual clones in MRS media supplemented with 1 mM glyphosate as described above. The cleared supernatants are subjected to GC-MS and clones one close is used for genomic DNA isolation, using standard commercial kits (Qiagen). The recovered genomic DNAs are used as a template to amplify the 16S rDNA gene. The resulting amplicons are purified and subjected to DNA sequencing using the forward primer used for PCR. The resulting sequences are used to conduct BLAST searches against the NCBI database. The DNA sequence provided a best match to Lactobacillus plantarum HA-119, consistent with the presence of the said bacterium in the original probiotic mixture.

TABLE 5

| | +L. plantarum | −L. plantarum |
|---|---|---|
| Growth Medium | HA-119 | HA-119 |
| Remaining glyphosate Concentration | 50% | 100% |
| (of 1 mM glyphosate) | | |

As illustrated in Table 5, 50% of glyphosate is removed from solution in the presence of Lactobacillus plantarum HA-119, while no glyphosate is removed in the control not containing bacteria. Glyphosate removal is deemed significant (p<0.05) by an ANOVA one-way analysis of variance.

### Example 3

Example 3—Demonstration of removal of glyphosate by Bacillus coagulans from an aqueous environment.

MRS liquid medium is inoculated with and without Bacillus coagulans NTCC-5856 supplemented with 1 mM glyphosate and is incubated overnight (12 hrs.) at 37° C. with shaking. The cultures are then transferred to a sterile microcentrifuge tube and cells are pelleted at 13,000 rpm for 3 minutes. An aliquot of the liquid medium is removed for analysis of the glyphosate concentration by GC-MS.

TABLE 6

| Growth Medium | +B. coagulans | −B. coagulans |
|---|---|---|
| Remaining glyphosate Concentration | 23% | 100% |
| (of 1 mM glyphosate) | | |

As illustrated in Table 6, 77% of glyphosate is removed from solution by Bacillus coagulans while no glyphosate is removed in the control. Removal is deemed significant (p<0.05) by an ANOVA one-way analysis of variance.

### Example 4

Example 4—Demonstration that removal of glyphosate from an aqueous environment is unique to some but not all probiotic combinations.

MRS liquid medium is inoculated with a mixture of probiotic bacteria containing Lactobacillus salivarius SD-5208 (and 12 other species of probiotic bacteria listed in Table 2; probiotic combination A), is supplemented with 1 mM glyphosate, and is incubated overnight (14 hrs.) at 37° C. with shaking. Additional probiotic mixtures are also tested for comparison Tables 7-9; probiotic combination B-D. The cultures are then transferred to a sterile microcentrifuge tube and cells are pelleted at 13,000 rpm for 3 minutes. An aliquot of the liquid medium is removed for analysis of the glyphosate concentration by GC-MS.

TABLE 7
List of bacterial species contained in Probiotic Combination B.

| Genus | Species | Strain |
|---|---|---|
| Bifidobacterium | lactis | Bl-04 |
| Lactobacillus | acidophilus | La-14 |
| Bifidobacterium | longum | Bl-05 |

TABLE 8
List of bacterial species contained in Probiotic Combination C.

| Genus | Species | Strain |
|---|---|---|
| Lactobacillus | acidophilus | La-04 |
| Bifidobacterium | longum | BB-536 |
| Bifidobacterium | lactis | Bl-05 |

TABLE 9
List of bacterial species contained in Probiotic Combination D.

| Genus | Species |
|---|---|
| Lactobacillus | salivarius |
| Lactobacillus | acidophilus |
| Lactobacillus | casei |
| Lactobacillus | plantarum |
| Lactobacillus | rhamnosus |
| Lactobacillus | bulgaricus |
| Bifidobacterium | breve |
| Bifidobacterium | longum |
| Bifidobacterium | bifidum |
| Bifidobacterium | lactis |
| Lactobacillus | paracasei |

Referring to FIG. 1, while probiotic combination, containing Lactobacillus salivarius reduced glyphosate levels Probiotic Bacteria Combination B, C and D did not remove glyphosate from an aqueous growth environment to any significant level. It is noteworthy that the probiotic mixture D, Table 9 contained Lactobacillus salivarius that did not contribute to glyphosate removal, suggesting that this activity is unique to some but not all strains of this species.

### Example 5

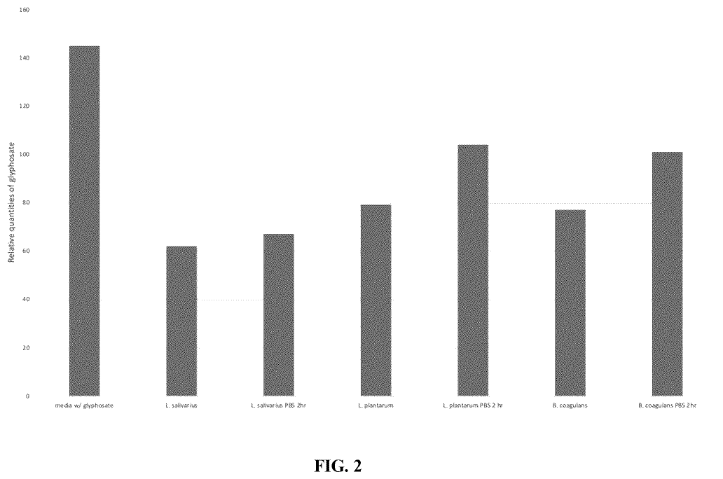

Example 5—To determine whether reductions in glyphosate in vitro are due to biochemical degradation or sequestration (adsorption), Lactobacillus salivarius SD-5208 Bacillus coagulans NTCC-5856, Lactobacillus plantarum HA-119 are cultured individually in MRS medium with 1 mM glyphosate or without added glyphosate for 14 hrs to saturation. The cultures containing glyphosate are centrifuged and supernatants are collected for GC-MS analysis to confirm previous results. Cultures not containing glyphosate are recovered by centrifugation and resuspended in phosphate buffered saline (PBS) containing 1 mM glyphosate. These mixtures are incubated for 2 hrs at 4° C. with agitation after which samples are centrifuged and supernatants are recovered for GC/MS analysis. Referring to FIG. 2, these experiments show nearly identical glyphosate reduction results as observed previously for actively growing cells. Given that saturated cultures are known to halt metabolism in bacterial cells, these are placed in PBS, so metabolic activity of cells is expected to remain essentially off. This coupled to a limited time frame and temperature for biochemical degradation to occur, we conclude that glyphosate reduction is due to sequestration of glyphosate most likely on the cell surface of strains.

### Example 6

Example 6—Mice fed with a diet containing glyphosate and food grade Lactobacillus salivarius SD-5208 Bacillus coagulans NTCC-5856, Lactobacillus plantarum HA-119, or mixtures containing these strains increase the levels of glyphosate in stool and decrease glyphosate levels in serum and liver samples.

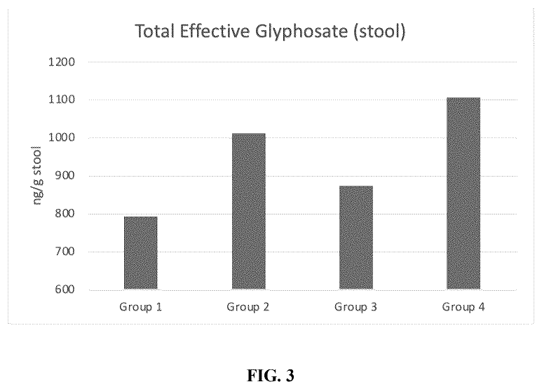

Referring to FIG. 3, C57Bl/6 wild-type laboratory mice, (n=48) are randomly selected to create 4 groups of mice (n=12). One group of mice (control Group 1) is given standard chow and water. All mice in the remaining 3 groups are provided standard chow and water with varying doses of the probiotic mixture. Group 2: 1×10⁶, group 3: 1×10⁷, group 4: 1×10⁸ cfu/day by oral gavage for 7 days. Urine, stool, liver and serum are collected at sacrifice on day 7. Glyphosate levels were measured in stool, urine, liver and serum as pools of 12 samples of equal mass or volume for all mice in each group. GC-MS analysis of glyphosate levels in stool samples show that prebiotic treated mice retained glyphosate at higher levels (not absorbed) compared to control mice.

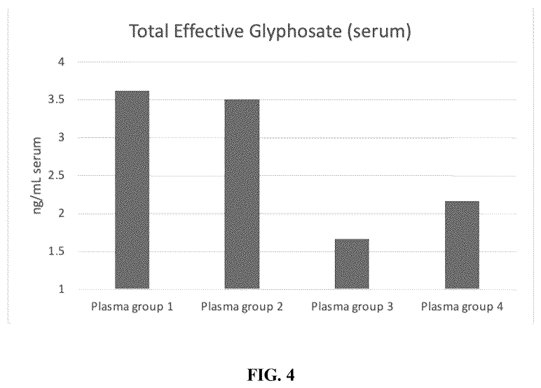

Referring to FIG. 4, mice receiving the highest dose of probiotic retained 40% more glyphosate compared to control mice. Measurements of serum glyphosate in mice from group 3 show a 46% reduction compared to control mice.

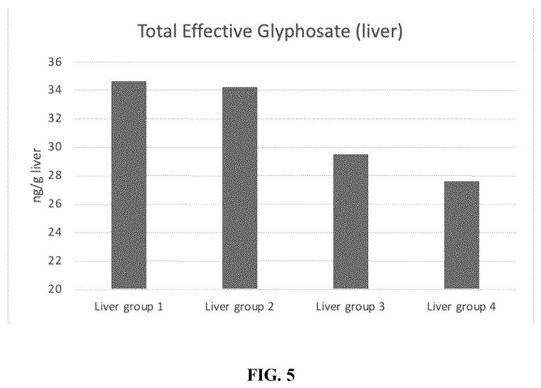

Referring to FIG. 5, measurements of liver glyphosate in mice from group 4 show a 20% reduction compared to control mice. While these values represent a marginal change considered “detoxification” the result is considered encouraging given that the probiotic intervention was carried out for just 1 week. It is our expectation that a longer intervention would result in larger decreases in liver glyphosate.

Finally, measurements of urine glyphosate in mice do not show significant differences in glyphosate as the result of probiotic treatment.

### Example 7

Example 7—Lactobacillus salivarius SD-5208 remove phthalates from aqueous solution in vitro.

To test whether Lactobacillus salivarius SD-5208 is also able to remove phthalates from solution in vitro, the previously identified strains are inoculated into MRS media supplemented with a 20 μM Di-n-butyl phthalate (DBP) or bisphenol A (BPA). These cultures are incubated overnight (14 hrs) at 37° C. with shaking. The cultures are transferred to a sterile microcentrifuge tube and cells are pelleted at 13,000 rpm for 3 minutes. The supernatants are then be subjected to GC-MS to determine whether phthalate concentrations are reduced.

TABLE 10

| Growth Medium | +L. salivarius | −L. salivarius |
|---|---|---|
| Remaining Concentration (of 20 μM | 50% | 100% |
| DBP) | | |

TABLE 11

| Growth Medium | +L. salivarius | −L. salivarius |
|---|---|---|
| Remaining Concentration (of 20 μM | 50% | 100% |
| BPA) | | |

As illustrated in Tables 10 and 11, 50% of phthalates, DBP and BPA are removed from solution by Lactobacillus salivarius while no phthalates are removed in the control. Removal is deemed significant (p<0.05) by an ANOVA one-way analysis of variance.

### Example 8

Example 8—Lactobacillus plantarum HA-119 remove phthalates from aqueous solution in vitro.

Lactobacillus plantarum HA-119 is tested to determine whether it is also able to remove phthalates from solution in vitro. The previously identified strain is inoculated into MRS media supplemented with a 20 μM Di-n-butyl phthalate (DBP) or bisphenol A (BPA). These cultures are incubated overnight (14 hrs) at 37° C. with shaking. The cultures are transferred to a sterile microcentrifuge tube and cells are pelleted at 13,000 rpm for 3 minutes. The supernatants are then be subjected to GC-MS to determine whether phthalate concentrations are reduced.

TABLE 12

| Growth Medium | +L. plantarum | −L. plantarum |
|---|---|---|
| Remaining Concentration (of 20 μM | 45% | 100% |
| BPA) | | |

As illustrated in Table 12, 55% of phthalates, DBP and BPA were removed from solution by Lactobacillus plantarum while no phthalates were removed in the control. Removal was deemed significant (p<0.05) by an ANOVA one-way analysis of variance.

### Example 9

Example 9—Bacillus coagulans remove phthalates from aqueous solution in vitro.

Bacillus coagulans is tested to determine whether it is also able to remove phthalates from solution in vitro. The previously identified strain is inoculated into MRS media supplemented with a 20 μM Di-n-butyl phthalate (DBP) or bisphenol A (BPA). These cultures are incubated overnight (14 hrs) at 37° C. with shaking. The cultures are transferred to a sterile microcentrifuge tube and cells are pelleted at 13,000 rpm for 3 minutes. The supernatants are then be subjected to GC-MS to determine whether phthalate concentrations are reduced.

TABLE 13
Growth Medium

| | +B. coagulans | −B. coagulans |
|---|---|---|
| Remaining Concentration (of 20 μM | 55% | 100% |
| BPA) | | |

As illustrated in Table 13, 45% of phthalates, BPA are removed from solution by Bacillus coagulans while no phthalates are removed in the control. Removal is deemed significant (p<0.05) by an ANOVA one-way analysis of variance.

### Example 10

Example 10—BPA binding protocol.

The concentration of BPA stock solution is based on the relative solubility of toxins in H₂O. The concentration of each toxin is based on available studies estimating human dietary exposure (Connolly A., Coggins M A, Koch, HM (2020) Human biomonitoring of glyphosate exposures: state-of-the-art and future challenges Toxics 8 (3) 60; Gari, M et al., (2023) Human-Biomonitoring derived exposure and Daily Intakes of Bisphenol A and their associations with neurodevelopmental outcomes among children of the Polish Mother and Child Cohort Study Environ Health 22 (1) 24) and set to represent the high end of those estimates.

Testing Live Bacteria for Toxin Binding Capacity. Starting from a single purified colony, or colony purified stock material, inoculate 3 ml MRS medium and incubate overnight at 37° C. with shaking. Incubation at 37° C. mimics the human body temperature that the material under test must eventually be proven to work in via the finished commercial product. Distribute 3 ml MRS media into 15 ml sterile conical tubes. Add Standardized Toxins Diluent Stock (see below). A culture containing strain of interest without added toxin is included. This supernatant is used to incubate toxin in spent medium to mimic experimental samples to serve as a Toxin Negative Control and reference for comparison to cultures containing toxin and cells.

### Standardized Toxins Diluent Stock Solution Preparation.

Given the small quantities of materials (10-25 mg), water is added to the quantity of BPA (Sigma Aldrich) material shown by the manufacturer to achieve the desired concentration of BPA stocks, as shown in Table 16 below. Materials are transferred to 50 ml plastic conical tubes and stored at −20° C. Because the shelf life of stocks has not been determined, the best practice is to generate standards from dry chemicals fresh for GC-MS or HPLC testing to allow detection of experimental material stability over time.

TABLE 14
BPA test stock solution.

| Toxin | Solvent | Stock | Dilution | [Final] |
|---|---|---|---|---|
| BPA | H₂O | 10 mM | 250X | 40 μM |

Ten microliters (μl) of overnight cultures are inoculated into 2 mL fresh MRS medium in 15 ml sterile conical test tube containing 40 μM BPA. Cultures are incubated overnight at 37° C. (with shaking) for 14-16 hours. One mL of binding reactions is transferred into a fresh sterile Eppendorf® microcentrifuge tubes and centrifuge at 13,000 rpm for 3 minutes. Taking care not to disturb the pellet, ˜800 μl of supernatant (spent medium) was transferred into fresh tubes. Samples were then subjected to quantification using GC-MS.

### Results

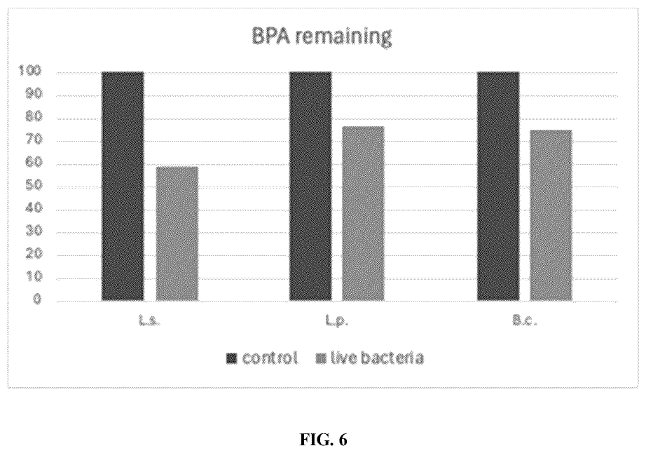

Referring to FIG. 6, all tested probiotic strains display BPA removal activity. L. salivarius removed over 41% of input BPA, whereas L. plantarum removed 24% and B. coagulans removed 25.2%.

To determine whether these isolates retained binding activity after heat-treatment, these were tested as follows.

### Determine Heat-Killed Bacteria for BPA Binding Capacity.

Starting from a single purified colony, or colony purified stock material, or culture house sourced single strain culture, inoculate sufficient MRS media to allow 2 mL of culture/condition. and incubate overnight at 37° C. (with shaking). Aliquots of culture were transferred to individual tubes and incubated in a water bath at 70° C. for 15 minutes to heat kill the bacteria. Distribute 1 ml of heat-killed culture into 2 ml sterile microcentrifuge tubes. BPA was added to heat-treated cultures to achieve a final concentration of 40 μM. One tube was reserved to contain spent supernatant without heat treated cells containing 40 μM BPA. Experimental and control mixtures were incubated at 37° C. (with shaking) for 14-16 hours. The mixtures are then placed into a centrifuge at 13,000 rpm for 3 minutes to pellet cell material. Taking care not to disturb the pellet, ˜800 of supernatant (spent medium) is transferred into fresh tubes for GC-MS analysis.

### Results

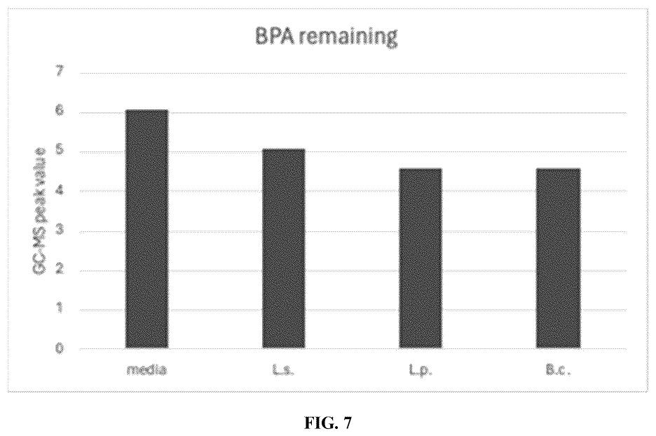

To determine whether the probiotic strains retained binding activity after heat-treatment at 70° C. for 15 minutes. Cultures in this example are performed in triplicate. All tested probiotic strains display BPA removal activity as post-biotics (See FIG. 7). Heat-treated L. salivarius removed over 17% of input BPA, whereas L. plantarum and B. coagulans removed 25%. These results are comparable to those obtained with live bacteria, albeit smaller quantities are removed. The standard deviation for triplicate measurements were as follows: control SD=0.67, L.s. SD=0.16, L.p. SD=. 512, B.c. SD=0.42.

Real world context of binding activities. We determined that 3 isolates screened for glyphosate binding activity removed 36-60% of input glyphosate. These results are confirmed by replicate experiments. This level of binding is equivalent to 26.5-63.8 μg of glyphosate removal and is considered of high significance given that it has been estimated that the average human may consume 40-240 μg of glyphosate daily. We conclude that a human probiotic dose (10-100 times more cells) is capable of removing virtually all consumed glyphosate.

Using the same 3 strains that bound glyphosate, we observed that 24-41% of input BPA was removed by live bacteria and 16-25% (heat-treated) of input BPA was removed from solution. Average human exposures to BPA are estimated to be 3.7 micrograms/day (μg/day). These findings are significant as they represent binding potentials equivalent to 100->200% of daily intake. These results obtained with both live and heat-killed bacteria indicate that both probiotic and postbiotic formulations are useful for BPA mitigation.

All references cited herein are incorporated by reference in their entireties. The present invention may be embodied in other specific forms without departing from the spirit or essential attributes thereof and, accordingly, reference should be made to the appended claims, rather than to the foregoing specification, as indicating the scope of the invention.

We claim:

1. A composition comprising a food-grade bacteria and a suitable carrier, wherein the composition comprises an effective dose of the food-grade bacteria to remove glyphosate and other toxic compounds from a substance or environment to which the food-grade bacteria is exposed.

2. The composition of claim 1, wherein the therapeutically effective dose is at least about 1×10⁸ of the food-grade bacteria per milliliter or less of the suitable carrier.

3. The composition of claim 2, wherein the suitable carrier is a carbohydrate-containing medium.

4. The composition of claim 3, wherein the carbohydrate-containing medium is a milk-based product.

5. The composition of claim 4, wherein the food-grade bacteria are provided dead or alive.

6. The composition of claim 5, wherein the food-grade bacteria are provided as an extract.

7. The composition of claim 6, wherein the composition comprises a combination of two or more different species of food-grade bacteria.

8. The composition of claim 7, wherein the composition comprises a combination of two or more strains of microorganisms selected from the group consisting of Lactobacillus salivarius SD-5208, Bacillus coagulans NTCC 5856, and Lactobacillus plantarum HA-119.

9. The composition of claim 8, wherein the environment is an aqueous environment.

10. The composition of claim 9, wherein said toxic compound is selected from the group consisting of glyphosate, phthalates, and plasticizers.

11. The composition of claim 10, wherein said plasticizer is bisphenol A (BPA).

12. A composition comprising food-grade bacteria, a carrier, and an animal's feed,
wherein said food-grade bacteria is capable of removing a toxic compound from a substance or environment to which the food-grade bacteria is exposed, and
wherein the food-grade bacteria comprises a bacterial isolate selected from the group consisting of the food-grade bacteria selected from the group consisting of Lactobacillus salivarius SD-5208, Bacillus coagulans NTCC 5856, and Lactobacillus plantarum SD-HA-119, or any combination thereof.

13. A method for reducing a subject uptake of toxic compounds consumed by the subject, comprising the step of administering to the subject an effective dose of a food-grade bacteria capable of sequestering the toxic compound consumed by the subject.

14. The method of claim 13, wherein the food-grade bacteria are provided dead or alive.

15. The method of claim 14, wherein the food-grade bacteria are provided as an extract.

16. The method of claim 15, wherein the food-grade bacteria comprise a combination of two or more different species of food-grade bacteria.

17. The method of claim 16 wherein the food grade bacteria are selected from the group of food-grade bacteria consisting of Lactobacillus salivarius SD-5208, Bacillus coagulans GG-828, and Lactobacillus plantarum SD-5209, or any combination thereof.

18. The method of claim 17, wherein said toxic compounds are selected from the group consisting of glyphosate, phthalates, and plasticizers.

19. The method of claim 18, wherein said glyphosate is a commercially available glyphosate containing herbicide formulation.

20. The method of claim 18, wherein said plasticizer is bisphenol A (BPA).

21. A method for removing a toxic compound from a substance or environment which is contaminated or suspected of being contaminated with said toxic compound comprising the step of contacting the substance or environment with food-grade bacteria capable of removing the toxic compound from the substance or the environment.

22. The method of claim 21, wherein the food-grade bacteria are provided dead or alive.

23. The method of claim 22, wherein the food-grade bacteria are provided as an extract.

24. The method of claim 23, wherein the food-grade bacteria comprise a combination of two or more different species of food-grade bacteria.

25. The method of claim 24, wherein the composition comprises a combination of two or more food grade bacteria selected from the group of food-grade bacteria consisting of Lactobacillus salivarius SD-5208, Bacillus coagulans NTCC 5856, and Lactobacillus plantarum SD-5209, or any combination thereof.

26. The method of claim 25 wherein the toxic compound is selected from glyphosate, phthalates, and plasticizers.

27. The method of claim 26 wherein said glyphosate is a glyphosate containing herbicide formulation.

28. The method of claim 26 wherein said plasticizer is bisphemol A (BPA).

29. A method of reducing the toxic effects of glyphosate in a subject, comprising the step of administering to the subject a therapeutically effective amount of a food-grade bacteria capable of removing the glyphosate from a substance or environment.

30. The method of claim 29, wherein said glyphosate is a commercially available glyphosate containing herbicide formulations.

31. The method of claim 30, wherein the food-grade bacteria are provided dead or alive.

32. The method of claim 31, wherein the food-grade bacteria are provided as an extract.

33. The method of claim 32, wherein the food-grade bacteria comprise a combination of two or more different species of food-grade bacteria.

34. The method of claim 33, wherein the composition comprises a combination of two or more food grade bacteria selected from the group of food-grade bacteria consisting of Lactobacillus salivarius SD-5208, Bacillus coagulans NTCC 5856, and Lactobacillus plantarum HA-119, or any combination thereof.

35. A method of obtaining a strain of food-grade bacteria from parental strains selected from the group of food-grade bacteria consisting of Lactobacillus salivarius SD-5208, Bacillus coagulans NTCC 5856, and Lactobacillus plantarum HA-119, wherein said food grade-bacteria are capable of removing a toxic compound from an environment, the method comprising the step of mutagenesis or genetically altering the bacteria.

36. The method of claim 35, wherein said toxic compound is selected from the group consisting of glyphosate, phthalates, and plasticizers.

37. The method of claim 36, wherein said plasticizer is bisphenol A (BPA).

38. A method for obtaining a cell fraction capable of removing a toxic compound from an environment, comprising the steps of:
a) culturing food-grade bacteria strains selected from the group of food-grade bacteria consisting of Lactobacillus salivarius SD-5208, Bacillus coagulans GG-828, and Lactobacillus plantarum SD-5209, or any combination thereof, and
b) recovering the cell fraction from the culture in step a).

39. The method of claim 38, wherein the food-grade bacteria are provided dead or alive.

40. The method of claim 39, wherein the food-grade bacteria are provided as an extract.

41. The method of claim 40, wherein the food-grade bacteria comprise a combination of two or more different strains.

42. The method of claim 41, wherein said toxic compound is selected from the group consisting of glyphosate, phthalates, and plasticizers.

43. The method of claim 42 wherein the plasticizer is bisphenol A (BPA).
